(19) United States
(12) Patent Application Publication
(10) Pub. No.: US 2025/0377351 A1
(43) Pub. Date: Dec. 11, 2025

(54) METHOD FOR IDENTIFYING EFFECTIVE TREATMENTS AGAINST NEURODEGENERATIVE DISORDERS

(71) Applicant: AcuraStem Incorporated, Pasadena, CA (US)

(72) Inventors: Justin K. ICHIDA, Pasadena, CA (US); Hongyan ZHOU, Pasadena, CA (US); Wen-Hsuan CHANG, Pasadena, CA (US); Samuel V. ALWORTH, Pasadena, CA (US)

(21) Appl. No.: 19/016,722

(22) Filed: Jan. 10, 2025

Related U.S. Application Data
(63) Continuation of application No. 16/732,109, filed on Dec. 31, 2019, now abandoned.
(60) Provisional application No. 62/786,823, filed on Dec. 31, 2018.

(51) Int. Cl. G01N 33/50; C12N 5/0793

(52) U.S. Cl. CPC G01N 33/5058; C12N 5/0619; C12N 2501/60; C12N 2506/1307; C12N 2506/45; G01N 2800/52

(57) ABSTRACT

The present invention relates to systems and methods for reprogramming cells to generate neurons and identifying effective treatments for neurodegenerative disorders using such neurons, as well as systems and methods for treating and developing treatments for one or more neurodegenerative disorders.

### CROSS-REFERENCE TO RELATED APPLICATIONS

This application is a continuation of U.S. patent application Ser. No. 16/732,109, filed Dec. 31, 2019, which claims the benefit of U.S. Provisional Application No. 62/786,823, filed Dec. 31, 2018, the entire contents of which are hereby incorporated by reference.

### FIELD OF THE INVENTION

The present invention relates to systems and methods for reprogramming cells to generate neurons and identifying effective treatments for neurodegenerative disorders using such neurons, as well as systems and methods for treating and developing treatments for one or more neurodegenerative disorders.

### BACKGROUND OF THE INVENTION

Many neurodegenerative disorders in patients are difficult to effectively treat, especially where the pathology of a neurodegenerative disorder in a particular patient is not completely understood.

Hundreds of distinct neuronal types are generated during the development of the vertebrate central nervous system (CNS), establishing a cellular diversity that is essential for the formation of neuronal circuits. The selective degeneration of specific types or classes of CNS neurons underlies many neurological disorders. This realization has generated interest in defining populations of progenitor cells that may serve as replenishable sources of neurons, with a view to treating neurodegenerative disorders. Directing such progenitor cells along specific pathways of neuronal differentiation in a systematic manner has proved difficult, not merely because the normal developmental pathways that generate most classes of CNS neurons remain poorly defined.

During development, neural cells are generated from embryonic stem cells through a series of developmental steps involving the regulation of signaling factors that impart to the stem cells a particular directional or positional character. Initially, ectodermal cells may acquire a rostral or caudal neural character, and differentiate into rostral or caudal neural progenitor cells, through the regulation of rostralizing and/or caudalizing embryonic signaling factors. Thereafter, the neural progenitor cells may differentiate further, acquiring the identity of a subtype of progenitor cells, or becoming a fully-differentiated neural cell, in response to the action of dorsalizing and/or ventralizing embryonic signaling factors.

Spinal motor neurons represent one CNS neuronal subtype for which many of the relevant pathways of neuronal specification have been defined (Jessell et al., Neuronal specification in the spinal cord: inductive signals and transcriptional codes. Nat. Rev. Genet., 1:20-29, 2000; Lee et al., Transcriptional networks regulating neuronal identity in the developing spinal cord. Nat. Neurosci., 4 Suppl.: 1183-91, 2001). The generation of spinal motor neurons appears to involve several developmental steps. Initially, ectodermal cells acquire a rostral neural character—a process achieved through the regulation of BMP, FGF, and Wnt signalling (Munoz-Sanjuan et al., Neural induction, the default model and embryonic stem cells. Nat. Rev. Neurosci., 3:271-80, 2002; Wilson et al., Neural induction: toward a unifying mechanism. Nat. Neurosci., 4 Suppl.: 1161-68, 2001). These rostral neural progenitors acquire a spinal positional identity in response to caudalizing signals that include retinoic acid (RA) (Blumberg et al., An essential role for retinoid signaling in anteroposterior neural patterning. Development, 124:373-79, 1997; Durston et al., Retinoids and related signals in early development of the vertebrate central nervous system. Curr. Top. Dev. Biol., 40:111-75, 1998; Muhr et al., Convergent inductive signals specify midbrain, hindbrain, and spinal cord identity in gastrula stage chick embryos. Neuron, 23:689-702, 1999). Subsequently, spinal progenitor cells acquire a motor neuron progenitor identity in response to the ventralizing action of Sonic Hedgehog protein (SHh) (Briscoe et al., Specification of neuronal fates in the ventral neural tube. Curr. Opin. Neurobiol., 11:43-49, 2001).

Several methods for generating neurons has been described in the literature. International Publication No. WO 2014/201133 describes methods for inducing the generation of neurons from non-neuronal cell types, for example, by contacting the cell or cell culture medium with one or more agents which inhibit Activin and/or PLKl signaling. International Publication No. WO 2017/117571 discloses methods for production of cortical neurons and motor neurons from certain cell lines (e.g., human embryonic stem cells (hESCs) or human induced pluripotent stem cells (hiPSCs)). “Clinical Trials in a Dish: The Potential of Pluripotent Stem Cells to Develop Therapies for Neurodegenerative Diseases” (https://www.ncbi.nlm.nih.gov/pmc/articles/-PMC4868344/) describes the morphogen directed differentiation of patient induced pluripotent stem cells to form neurons. “iPS cells: a game changer for future medicine” (https://www.embopress.org/doi/full/10.1002/embj.201387098) recognizes that one approach to improve on morphogen directed differentiation of iPSCs is to adopt methods to induce transcription factors for direct differentiation, i.e. transcription factor mediated reprogramming, which can be used to induce specific types of cells, including neurons.

Physicians often select treatments based on the symptoms exhibited by a patient and their own prior experience with various treatments. This often results in the selection of an ineffective or sub-optimal treatment.

Effective treatments for amyotrophic lateral sclerosis (ALS) have been particularly difficult to achieve. Although antisense therapies targeting causal mutations are under development for rare forms of amyotrophic lateral sclerosis (ALS), over 80% of ALS cases are sporadic, are not caused by known mutations, and likely result from diverse genetic etiologies. Thus, new therapeutic strategies for sporadic ALS are needed.

International Publication No. WO 2016/210372 discloses a method of treating a neurodegenerative disease by administering a PIKFYVE inhibitor.

There remains a need for improved methods for selecting treatments for neurogenerative disorders.

### SUMMARY OF THE INVENTION

The present invention provides improved methods for selecting effective treatments for patients having neurodegenerative disorders, such as amyotrophic lateral sclerosis (ALS).

One embodiment is a method for identifying a suitable treatment for a patient having a neurodegenerative disorder comprising: (i) generating neurons from cells (for example, from fibroblasts or from induced pluripotent stem cells which are generated from somatic cells) obtained from a patient; (ii) testing two or more prospective treatments against separate groups of the generated neurons; and (iii) assessing the neurons following each treatment. The prospective treatments may be selected from a library of treatments for the neurodegenerative disorder, including known treatments (such as those approved by a regulatory authority (e.g., the U.S. Food and Drug Administration) or considered the standard of care), experimental treatments (e.g., treatments which are the subject of a clinical trial for the neurodegenerative disorder), and other treatments (i.e., treatments not known and not currently the subject of a clinical trial for the neurodegenerative disorder). In one embodiment, the assessment step (iii) includes determining the survival rate of neurons following each prospective treatment. The survival rate can be assessed by imaging. The method may further include (iv) identifying one or more suitable treatments based on the assessment in step (iii), and optionally (v) treating the patient with an identified suitable treatment from step (iv).

In one embodiment, the neurons in step (i) are generated within 2 months, 6 weeks, 1 month, or 3 weeks.

In another embodiment, the neurons are motor neurons. The motor neurons can be generated from fibroblasts and/or pluripotent stem cells. Preferably, the motor neurons are generated in an efficient and cost-effective manner. In one embodiment, the neurons are generated from iPSCs or fibroblasts obtained from a patient.

In another embodiment, the neurons in step (i) are generated from fibroblasts obtained from a patient, where the fibroblasts have been reprogrammed to produce neurons in higher yield. The fibroblasts may be reprogrammed with at least one transcription factor and optionally a TGF-beta inhibitor, a Ras mutant, a p53 mutant lacking a DNA-binding domain, or any combination of any of the foregoing.

Yet another embodiment is a method for evaluating a prospective treatment for a patient having a neurodegenerative disorder comprising:
(i) generating neurons from fibroblasts obtained from a patient, wherein the fibroblasts have been reprogrammed to produce neurons in higher yield;
(ii) testing the prospective treatment against the generated neurons;
(iii) assessing the neurons following treatment; and
(iv) determining whether to treat the patient with the prospective treatment based on the assessment in step (iii). In one embodiment, the prospective treatment is the subject of a clinical trial, and step (iv) includes determining whether the patient should be a subject in the clinical trial.

Yet another embodiment is a method for selecting patients for a clinical trial involving a prospective treatment for a neurodegenerative disorder. The method involves for each patient (i) generating neurons from fibroblasts or from induced pluripotent stem cells which are generated from somatic cells obtained from the patient; (ii) testing the prospective treatment against the generated neurons; (iii) assessing the neurons following treatment; and (iv) selecting one or more patients for the clinical trial based on the results of the assessment in step (iii).

The methods described herein are particularly useful for rapidly progressive neurodegenerative disorders.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE FIGURES

A more complete appreciation of the invention and many of the attendant advantages thereof will be readily obtained as the same becomes better understood by reference to the following detailed description when considered in connection with the accompanying drawings, wherein:

FIG. 1 shows immunofluorescence images demonstrating the reprogramming efficiency of fetal and adult human fibroblasts into neurons;

FIG. 2A-H show 7 factor reprogramming of iPSCs in the generation of mature, disease-relevant induced motor neurons (iMNs);

FIG. 2A shows immunocytochemical images where iMNs express the Hb9::RFP reporter (red), TUJ1 (yellow), HB9 (green). Nuclei, Hoechst (blue). Scale bars: 125 μm;

FIG. 2B shows the number of control and C9-ALS iMNs generated per 0.5 mm² of culture dish area. Mean±s.d. n=6 independent iMN conversions per line;

FIG. 2C shows the quantification of iMN generation, showing the amount of HB9+ cells as a proportion of HB9::RFP+ cells, or of DAPI+ nuclei, or of MAP2+ cells. Mean±s.d. n=3 (for HB9/Hb9::RFP and HB9/DAPI) or 4 (HB9/MAP2) independent iMN conversions;

FIG. 2D-2F show representative patch clamp recordings showing that iMNs generated by iPSC-7F, fibroblast 7F, and fibroblast 7F-DD as described herein, possess functional sodium and potassium channels and fire action potentials spontaneously;

FIG. 2G shows gene expression analysis of postmortem and patient iMN samples. Samples used were control iMNs, C9ORF72 ALS patient iMNs, C9ORF72+/−iMNs (one copy of C9ORF72 deleted), frontal cortex from C9ORF72 and sporadic ALS patients (GSE67196), and laser capture microdissected motor neurons from C9ORF72 and sporadic ALS patients (GSE56504). Sporadic patient data is included as a reference. n=number of consensus genes analyzed, which were identified by DESeq2 analysis as being significantly differentially expressed (p<0.05) in all postmortem C9ORF72 patient datasets;

FIG. 2H shows gene expression analysis of postmortem and patient iMN samples. Samples used were control iMNs, C9ORF72 ALS patient iMNs, C9ORF72+/−iMNs (one copy of C9ORF72 deleted), frontal cortex from C9ORF72 and sporadic ALS patients (GSE67196), and laser capture microdissected motor neurons from C9ORF72 and sporadic ALS patients (GSE56504). Sporadic patient data is included as a reference. n=number of consensus genes analyzed, which were identified by DESeq2 analysis as being significantly differentially expressed (p<0.05) in all postmortem C9ORF72 patient datasets;

FIG. 3A-H show C9ORF72 and sporadic ALS iPSC-7F iMNs display ALS phenotypes;

FIG. 3A shows the production of Hb9::RFP+iMNs and survival tracking by time-lapse microscopy;

FIG. 3B shows the survival of control (CTRL) and C9ORF72 ALS patient (C9-ALS) iMNs with a 12-hour pulse treatment of excess glutamate (shown for each individual line separately; n=90 iMNs per line for 3 control and 2 C9-ALS lines;

FIG. 3C shows the survival of control and sporadic ALS (sALS) patient lines after glutamate treatment (iMNs from all control or C9ORF72 patient lines shown in aggregate. n=90 iMNs per line for 3 control and 6, except sALS6 which had 60 iMNs counted. iMNs quantified from 3 biologically independent iMN conversions per line.

FIG. 3D shows quantification to determine endogenous poly (GR)+ punctae in control or C9-ALS iMNs. Quantified values represent the average number of nuclear poly (GR)+ punctae in n=30 iMNs (controls) or 40-44 iMNs (C9-ALS) per line per condition from two control or two C9-ALS patient lines. For each line, iMNs were quantified from two independent iMN conversions per line per condition. Median+/−interquartile range. Each grey circle represents the number of poly (GR)+ punctae/unit area in a single iMN. Mann-Whitney testing;

FIG. 3E shows immunostaining to determine endogenous poly (GR)+ punctae in control or C9-ALS iMNs. Quantified values represent the average number of nuclear poly (GR)+ punctae in n=30 iMNs (controls) or 40-44 iMNs (C9-ALS) per line per condition from two control or two C9-ALS patient lines. For each line, iMNs were quantified from two independent iMN conversions per line per condition. Median+/−interquartile range. Each grey circle represents the number of poly (GR)+ punctae/unit area in a single iMN. Solid and dotted lines in FIG. 4E outline the cell body and nucleus, respectively. Scale bar=5 m. Scale bar=5 m;

FIG. 3F shows immunofluorescence analysis of total TDP-43;

FIG. 3G shows quantification of the ratio of nuclear to cytoplasmic total TDP-43 in control, C9-ALS. Median+/−interquartile range. Kruskal-Wallis testing;

FIG. 3H shows quantification of the ratio of nuclear to cytoplasmic total TDP-43 in control, sporadic ALS iMNs. Ratio of nuclear to cytoplasmic total TDP-43 in individual C9-ALS iMNs. iMNs from two controls, two C9-ALS, and four sporadic ALS patients. were quantified. n=30 iMNs per line (control and C9-ALS) per condition or n=26 iMNs, 30 iMNs per condition per line from two biologically independent iMN conversions were quantified. Each grey circle represents a single iMN. For FIG. 4H, mean+/−s.e.m. Unpaired t-test with Welch's correction. Scale bars=5 μm. Dotted lines outline the nucleus and cell body. The day of differentiation stated on each panel indicates the day of differentiation on which the experimental treatment or time course was initiated;

FIG. 4A-J show chemical and genetic cocktail enables near-deterministic reprogramming of fibroblasts into motor neurons;

FIG. 4A shows 6F iMNs, 14 days post infection (dpi). Scale bar=100 μm;

FIG. 4B shows 6FDDRR-iMNs, 14 dpi. Scale bar=100 μm;

FIG. 4C shows iMN yield in 6F, 6FDD, or 6FDDRR conditions at 14 dpi. Conversion yield determined by counting Hb9::GFP+ cells with neuronal morphology divided by number of cells seeded. n=10-20 independent conversions per condition. Mean±/−s.e.m;

FIG. 4D shows one-way ANOVA Hb9::GFP+ cells with fibroblast morphology at 17 dpi. Scale bars=20 μm;

FIG. 4E shows one-way ANOVA Hb9::GFP+ cells with neuronal morphology at 17 dpi. Scale bars=20 μm;

FIG. 4F shows the Percentage of Hb9::GFP+ cells with neuronal morphology of total Hb9::GFP+ cells at 17 dpi. n=9 independent conversions per condition. Mean±/−s.e.m. One-way ANOVA;

FIG. 4G shows a dot plot of CFSE intensity and fluorescently labeled-EU for Control-Puro (grey), 6F (green), and 6FDDRR (red). Histograms of CFSE and EU intensity adjacent to dot plot. Quadrant to demark hypertranscribing, hyperproliferating cells (HHCs) set by reference to 6F condition. Hyperproliferating and slow cycling cells set by selecting CFSE value in 6F condition to allow the dimmest 15%. High EU values set by top half of 6F condition, resulting in ˜7% HHCs in 6F;

FIG. 4H shows the percentage of HHCs. n=11-16 independent conversion per condition. Median+/−interquartile range. Kruskal-Wallis Test.

FIG. 4I shows the yield of Hb9::GFP+ cells counted via flow cytometry at 17 dpi normalized to number of seeded cells. n=7-8 independent conversions per condition. Mean±/−s.e.m. Unpaired t-test;

FIG. 4J shows the percentage of binucleated iMNs at 14 dpi. n=6 independent conversions. Mean±/−s.e.m. Unpaired t-test. Significance summary: p>0.05 (ns), *p≤0.05, ** p≤0.01, *** p≤0.001, and **** p≤0.0001;

FIG. 5A shows an exemplary method of neuron generation, in accordance with an embodiment of the invention;

FIG. 5B shows another exemplary method of neuron generation, in accordance with an embodiment of the invention;

FIG. 6 shows a summary of motor neuron generation and motor neuron pathology assessment, in accordance with an embodiment of the invention;

FIG. 7 shows an iPSC 7F iMNs survival assay that was established and proprietary automated image analysis technologies to determine the neuron survival rate;

FIG. 8A-8B show the identification and mechanism of PIKFYVE inhibition as a therapeutic target for ALS;

FIG. 8A shows a screen comprising over 1,100 therapeutics, where seven compounds that were previously used in an advanced phase were identified, randomized and controlled in a clinical trial with ≥100 ALS patients between 1995 and July 2016. Red indicates a failed clinical trial result, whereas orange indicates a mixed clinical trial result. Neurotrophic Factors and Apilimod were used as positive controls. The assay metric, the Hazard Ratio (hR), is calculated by using the Cox Proportional Hazards model comparing each condition to DMSO. P-values indicate test of significance at the 95% confidence level against the pooled DMSO aggregate;

FIG. 8B is a schematic showing the PIKFYVE inhibition mechanism of action;

FIG. 9A-E show how PIKFYVE inhibition rescues the survival of C9ORF72 and sporadic ALS iMNs;

FIG. 9A shows three C9-ALS iMN lines that were treated with 3 μM Apilimod for 2 weeks (media change every 3 days) during the survival assay. The hazard ratio was generated by comparing the Apilimod treatment condition to the DMSO condition in the corresponding C9-ALS line. Apilimod 3 μM showed a significant beneficial effect in all three C9-ALS lines. (P value: *<0.05, ***<0.0005);

FIG. 9B shows four sporadic ALS lines that were treated with 10 μM Apilimod for 3 weeks during the survival assay. (P value: ****<0.0001);

FIG. 9C shows five healthy control lines that were treated with 1 μM Apilimod for 3 weeks during the survival assay. No significance difference between treatment and DMSO group was observed;

FIG. 9D shows sALS iMNs that were treated with QJ999ASX from the AS-1 Series, dose response curves and EC50 values were fitted and calculated using Prism software. hR values from 4 of the doses shown in are plotted. hR and p values are calculated using the Cox proportional hazards model comparing each dose to DMSO (p values directly above the 95% CI). To evaluate inter dose significance, each ascending dose was compared to the prior dose using the Cox proportional hazards model and the resulting p-values are shown for each comparison (upper level). The comparison of a roughly 3 fold step size in compound concentration (300 nM to 1 uM) was found to be significantly different (p value=4.8e-5);

FIG. 9E shows qRT-PCR of PIKFYVE expression in iMN cultures treated with 3 μM scrambled or PIFKVYE ASOs for 72 hrs. One way-ANOVA with Tukey correction across all comparisons. n=6 biologically independent iMN conversions in each condition. Mean±/−s.d.;

FIG. 10A-H show how treatment with PIKFYVE inhibitors and other treatments such as an anticoagulation-deficient form of activated protein C, 3K3A-APC, rescues TDP43, C9 DPR, and motor function phenotypes in patient iMNs;

FIG. 10A-10C show immunofluorescence analysis of total DPRs and quantification of the number of DPRS per unit area of two healthy control and two C9ALS lines. For FIGS. 10B and 10C, Mann-Whitney testing was used;

FIG. D-G show immunofluorescence analysis of total TDP-43 and quantification of the ratio of nuclear to cytoplasmic of two healthy control, two C9 ALS, and two sporadic ALS lines, where iMNs were treated with 10 nM of the active or inactive form of 3K3A-APC and then stained against TDP-43 antibody. The nuclear to cytoplasmic area fluorescence intensity was measured by Image J. 30 iMNs were analyzed for each line. Results from each treatment condition were pooled together. The Mann-Whitney test was used

FIGS. 10H and 10I show the effect of 3 μM Apilimod on the level of GR+ puncta in the dentate gyrus of control or C9-BAC mice. Data are presented as mean±s.d. of the number of GR+ puncta per cell, each data point represents a single cell. n=20 (wild type+DMSO), 20 (wild type+Apilimod), 87 (C9-BAC+DMSO) and 87 (C9-BAC+Apilimod) cells quantified from three mice per condition, one-way ANOVA with Tukey correction for all comparisons, F value (DFn, DFd): (3, 180)=16.29. Scale bars represent 2 μm, dotted lines outline nuclei, and white arrows denote GR+ punctae; and

### DETAILED DESCRIPTION OF THE INVENTION

In describing a preferred embodiment of the invention illustrated in the drawings, specific terminology will be resorted to for the sake of clarity. However, the invention is not intended to be limited to the specific terms so selected, and it is to be understood that each specific term includes all technical equivalents that operate in a similar manner to accomplish a similar purpose. Several preferred embodiments of the invention are described for illustrative purposes; it being understood that the invention may be embodied in other forms not specifically shown in the drawings.

### Definitions

“C9ORF72” as used herein, refers to a protein which in humans is encoded by the gene C9orf72.

“Dipeptide repeat proteins” as used herein, refer to proteins that are generated from repeat-associated non-ATG (RAN) translation of mutant C9ORF72 transcripts.

“DPR,” as used herein, refers to dipeptide repeat proteins.

“Hyperproliferative cells” as used herein, refer to cells with a high rate of proliferation by rapid division.

“Lineage conversion,” as used herein, refers to the direct reprogramming of somatic cells into a target cell type by forced expression of reprogramming factors without going through an intermediate iPS cell state.

As used herein, the term “morphogen directed differentiation” refers to a manipulation of stem cell culture conditions to induce differentiation into a particular cell type, such as a spinal cord motor neuron, for example by using signaling compounds.

“Motor neuron,” as used herein, refers to a nerve cell forming part of a pathway along which impulses pass from the brain or spinal cord to a muscle or gland.

“Neurons” as used herein, refers to cells within the nervous system that transmit information to other nerve cells, muscle, or gland cells.

Unless otherwise indicated, the term “patient” refers to a mammal, such as a domestic animal (e.g., a cat or dog) or farm animal (e.g., a horse, cow, bull, sheep, or pig), and preferably refers to a human. A human patient can be of any age, including infants and toddlers up to 2 years of age, children from 2 to 12 years of age, adolescents (e.g., 12 to 18 years of age), and adults (18 years of age and older).

“PIKFYVE Inhibitor” as used herein, refers to a compound or molecule that interferes with (e.g., reduces, decreases, suppresses, eliminates, or blocks) the PIKFYVE enzyme activity. PIKFYVE is also known in the art as phosphatidylinositol-3-phosphate 5-kinase type III or PIPKIII.

“TDP43,” as used herein, refers to the TDP 43 protein and/or the gene that encodes it.

“Transcriptional factor mediated reprogramming” refers to the conversion of one cell type into another by forced expression of master transcription factors.

“WT,” as used herein, means “wild-type”.

“7F” and “7 factor protocol,” as used herein, refers to increasing the protein expression of 7 motor neuron inducing factors (Lhx3, Ascl1, Brn2, Myt1l, Isl1, Ngn2 and NeuroD1) by transcription factor mediated reprogramming.

### Neurodegenerative Disorder

A neurodegenerative disorder is any disorder or disease that causes electrical, biochemical, or structural abnormalities in the brain, spine, or neurons, where the disorder or disease results in the progressive destruction of neurons that affects neuronal signaling. The neurodegenerative disorder may result in motor neuron degeneration. Neurodegenerative disorders include, but are not limited to, Huntington's disease, Alzheimer's disease, dementia such as frontotemporal dementia and Lewy body dementia, Parkinson's disease, multiple sclerosis, amyotrophic lateral sclerosis (ALS), amyloid lateral sclerosis, Friedreich's ataxia, Parkinson's disease, spinal muscle atrophy, primary lateral sclerosis, progressive muscle atrophy, progressive bulbar palsy, pseudobulbar palsy, Creutzfeldt Jakob disease, corticobasal degeneration, and progressive supranuclear palsy.

The neurodegenerative disorder may be associated with aberrant lysosome degradation. Cathepsin imbalance during aging and age-related diseases may provoke deleterious effects on CNS neurons and lysosomes may be sites for the unfolding and partial degradation of membrane proteins or their precursors that subsequently become expelled from a cell, or are released from dead cells and accumulate as pathological entities.

A health care professional may diagnose a subject as having a disease associated with motor neuron degeneration by the assessment of one or more symptoms of motor neuron degeneration. To diagnose a neurological disease, a physical exam may be followed by a thorough neurological exam. The neurological exam may assess motor and sensory skills, nerve function, hearing and speech, vision, coordination and balance, mental status, and changes in mood or behavior. Non-limiting symptoms of a disease associated with a neurological disease may be weakness in the arms, legs, feet, or ankles; slurring of speech; difficulty lifting the front part of the foot and toes; hand weakness or clumsiness; muscle paralysis; rigid muscles; involuntary jerking or writing movements (chorea); involuntary, sustained contracture of muscles (dystonia); bradykinesia; loss of automatic movements; impaired posture and balance; lack of flexibility; tingling parts in the body; electric shock sensations that occur with movement of the head; twitching in arm, shoulders, and tongue; difficulty swallowing; difficulty breathing; difficulty chewing; partial or complete loss of vision; double vision; slow or abnormal eye movements; tremor; unsteady gait; fatigue; loss of memory; dizziness; difficulty thinking or concentrating; difficulty reading or writing; misinterpretation of spatial relationships; disorientation; depression; anxiety; difficulty making decisions and judgments; loss of impulse control; difficulty in planning and performing familiar tasks; aggressiveness; irritability; social withdrawal; mood swings; dementia; change in sleeping habits; wandering; change in appetite.

Tests may be performed to rule diseases and disorders that may have symptoms similar to those of neurodegenerative disorders, including measuring muscle involvement and assessing neuron degeneration. Non-limiting examples of tests are electromyography (EMG); nerve conduction velocity study; laboratory tests of blood, urine, or other substances; magnetic resonance imaging (MRI); magnetic resonance spectroscopy; muscle or nerve biopsy; transcranial magnetic stimulation; genetic screening; x-rays; fluoroscopy; angiography; computed tomography (CT); positron emission tomography; cerebrospinal fluid analysis; intrathecal contrast-enhanced CT scan; electroencephalography; electronystagmography; evoked response; polysomnogram; thermography; and ultrasound. A health care professional may also assess the patient's family history of diseases associated with motor neuron degeneration and make a diagnosis in part based on a familial history of neurological diseases. A healthcare professional may diagnose a disease associated with neurological disease in a subject after the presentation of one or more symptoms.

Diseases associated with motor neuron degeneration may be a condition that results in the progressive destruction of motor neurons that interferes with neuronal signaling to the muscles, leading to muscle weakness and wasting. In healthy individuals, upper motor neurons transmit signals from the brain to lower motor neurons in the brain stem and spinal cord, which then transmit the signal to the muscles to result in voluntary muscle activity. The destruction of upper and lower motor neurons affects activity such as breathing, talking, swallowing, and walking, and overtime these functions can be lost. Examples of motor neuron diseases include, but are not limited to, amyotrophic lateral sclerosis, primary lateral sclerosis, progressive muscle atrophy, progressive bulbar palsy, and pseudobulbar palsy. The etiology of disease associated with motor neuron degeneration has not been fully elucidated and has been attributed to genetic factors and sporadic cases.

Rapidly progressive neurodegenerative disorders include those where neurodegeneration can be observed by an objective measure, such as a CT scan, after a period of 30, 60, or 90 days. Certain forms of dementia, for instance, are rapidly progressive.

### Neuron Generation

Suitable methods for obtaining suitable cells from a subject for neuron generation include, but are not limited to, a skin biopsy or blood extraction.

Neuron generation commences with blood extraction from people with ALS. The blood cells are then converted back into an embryonic stem cell like state called human induced-pluripotent stem cells (hiPSCs).

Neuron generation may be performed using techniques such as morphogen directed differentiation, linear conversion, and any combination of any of the foregoing. For instance, induced pluripotent stem cells (iPSCs) may be differentiated into CNS cells of interest. Protocols have built on lessons from developmental studies that elucidated the combination and timing of instructive queues to drive differentiation of specific lineages. Approaches generally involve growth factors or small molecules to recapitulate the ontogeny of the cell type of interest, for example, neurons and astrocytes. This can also be accomplished by directly reprogramming somatic cells with ectopic expression of genetic drivers toward a given lineage, such as neural precursor cells, neurons, and astrocytes. Specific subtypes of CNS cells, including layer-specific cortical neurons, dopaminergic neurons, motor neurons, striatal neurons, and cortical interneurons, can also be derived.

One type of differentiation technique is morphogen directed differentiation. Morphogen directed differentiation involves the use of morphogens. Morphogens are diffusible molecules that elicit signaling responses in surrounding cells in a concentration dependent manner. They modulate gene expression in a graded manner to provide positional information in developing embryos to ensure correct body patterning and formation of body structures both in vertebrate and in invertebrate organisms.

Morphogen directed differentiation of hiPSCs can be performed using standard techniques, which may utilize, for example, growth factors to drive differentiation. A non-comprehensive list of such morphogens includes: CNTF, ciliary neurotrophic factor; d, days of growth factor-driven differentiation; EGF, epidermal growth factor; FGF, fibroblast growth factor; FGF8, fibroblast growth factor 8; GSK3βi, glycogen synthase kinase-3β inhibitor; iPSC, induced pluripotent stem cell; RA, retinoic acid; SHH, sonic hedgehog; SMAD, intracellular proteins that transduce extracellular signals from TGFβ signaling; WNT, family of Wnt signaling pathways; WNTi, inhibitors of Wnt signaling pathways.

Morphogen directed differentiation may involve directly reprogramming somatic cells with ectopic expression of genetic drivers toward a given lineage, such as neural precursor cells, neurons, and astrocytes.

Another differentiation technique is lineage conversion. Lineage conversion involves the direct reprogramming of somatic cells into a target cell type by forced expression of reprogramming factors without going through an intermediate iPS cell state is a powerful tool for disease modelling because of the ability to generate specific mature human somatic cells within a short period of time.

Ectopic gene expression techniques can be used to manipulate cell lineage in a dish, converting cells from one specialized phenotype to another. An early demonstration of this idea was an experiment showing that fibroblasts can be converted into cells displaying the characteristic features of muscle cells upon transfection with a synthetic plasmid construct expressing MyoD, a key regulator of myogenic development in vivo. This represents an engineered “transdifferentiation,” (i.e., a direct conversion of a somatic cell from one terminally-differentiated cell type to another). The genes which can be used to promote such lineage conversions are typically “transcription factors,” (i.e., they belong to the class of proteins, which interacts directly with DNA in a sequence-specific manner to regulate the expression of other genes). In some cases, genes encoding other types of proteins or certain non-coding RNAs such as microRNAs and long non-coding RNAs can also affect cell fate. Cell lineage conversion does not require indefinite transgene expression because the various naturally-occurring cell types represent stable “attractors” in gene expression space: once established, their underlying pattern of gene expression is self-reinforcing and refractory to ordinary perturbations. The ectopic expression of regulatory factors governing cell lineage is typically sustained for at least several days to activate a stable pattern of genetic regulatory network activity and remodel the epigenetic state of the chromatin sufficiently to effect a lasting change in cellular phenotype.

A non-exhaustive list of linear conversion techniques is provided below:
(1) Techniques based on excisable integrating vectors. In one approach, the use of lentiviral vectors featuring flanking recombination sites allows integrated transgenes to be edited out through a post-reprogramming cleanup step based on brief expression of a recombinase enzyme by transient transfection of a plasmid or messenger RNA. Another approach uses a transposon vector to embed transgene expression cassettes in the genome. After reprogramming, plasmid or mRNA transfection can be used to express a transposase enzyme to purge integrated transposon sequences from the genome.
(2) Techniques based on non-integrating DNA vectors. Variations involve the use of multiple rounds of plasmid transfection or, alternatively, one-shot transfection of an episome (i.e., a circular DNA featuring a eukaryotic origin of replication included to prolong transgene expression in dividing cells). Reprogramming can has also be performed using non-integrating adenoviral vectors.
(3) Techniques based on non-DNA expression vectors such as protein or RNA molecules, or viruses having completely RNA-based life cycles. This class include delivery of reprogramming factors in the form of recombinant proteins featuring cell membrane-penetrating peptide domains (referred to as “protein transduction”), transfection with synthetic mRNA or microRNA (or some combination of both), transfection of special self-replicating mRNA molecules that exploit features derived from alphaviruses, and the use of Sendai virus as an expression vector.

Typical lineage conversion techniques are also exemplarily described in “Direct Lineage Reprogramming: Strategies, Mechanisms, and Applications,” Cell Stem Cell, 16:2, February 2015, pgs. 119-134, which is hereby incorporated by reference in its entirety.

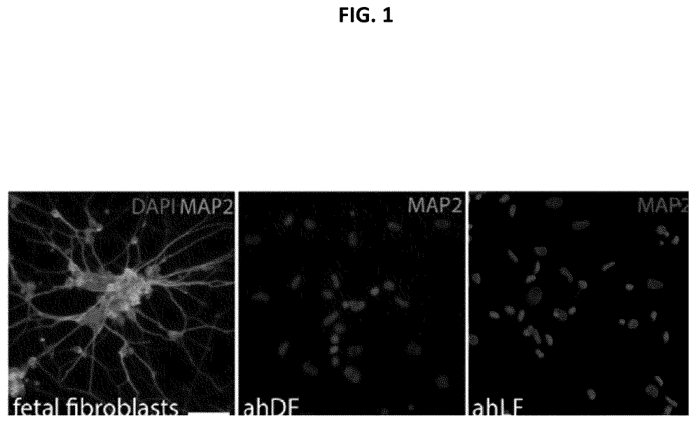

FIG. 1 shows the reprogramming efficiency of fetal and adult human fibroblasts into neurons. The immunofluorescence images show reprogramming efficiency with adult fibroblasts. As shown in FIG. 1, “ahDF” represents adult human dermal fibroblasts and “ahLF” represents adult human lung fibroblasts.

In one embodiment, neuron generation commences with blood extraction from a subject (e.g., a subject with ALS). The blood cells are then converted back into an embryonic stem cell like state called human induced-pluripotent stem cells (hiPSCs).

The cells may be converted into motor neurons, for example, by increasing the protein expression of 7 motor neuron inducing factors (i.e. Lhx3, Ascl1, Brn2, Myt1l, Isl1, Ngn2 or NeuroD1) by transcription factor mediated reprogramming (“the 7F reprogramming cocktail”).

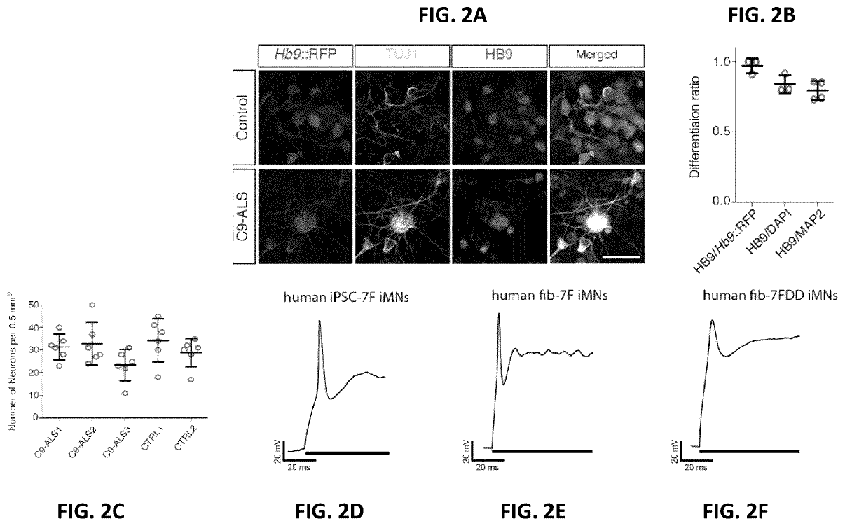

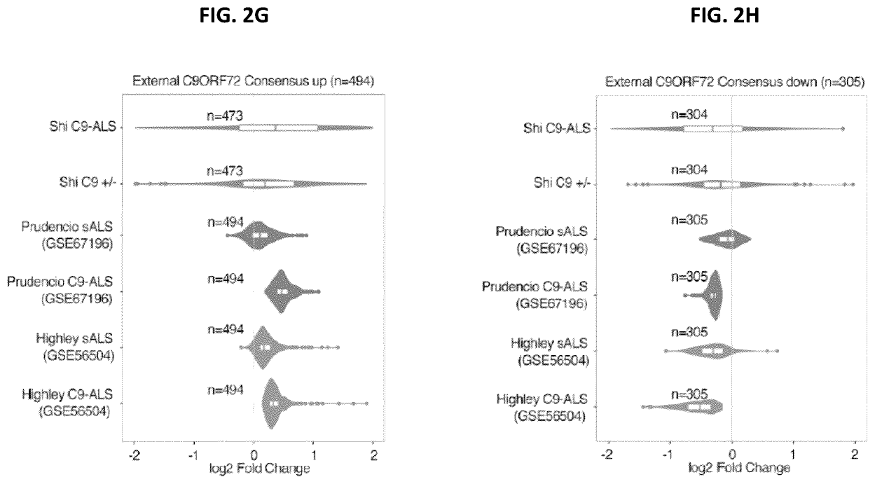

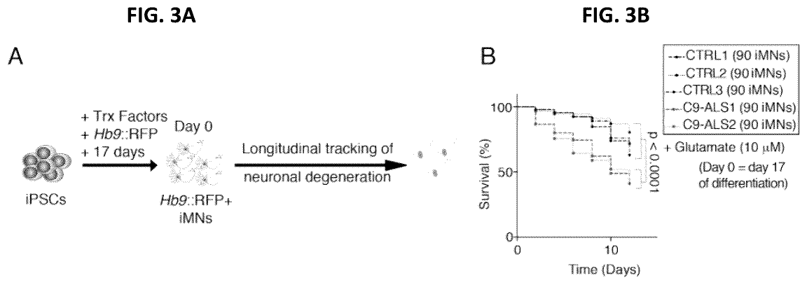

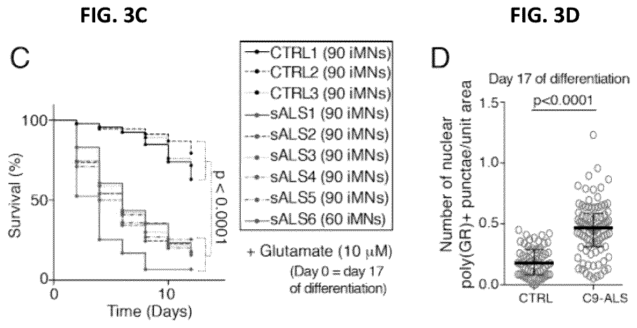

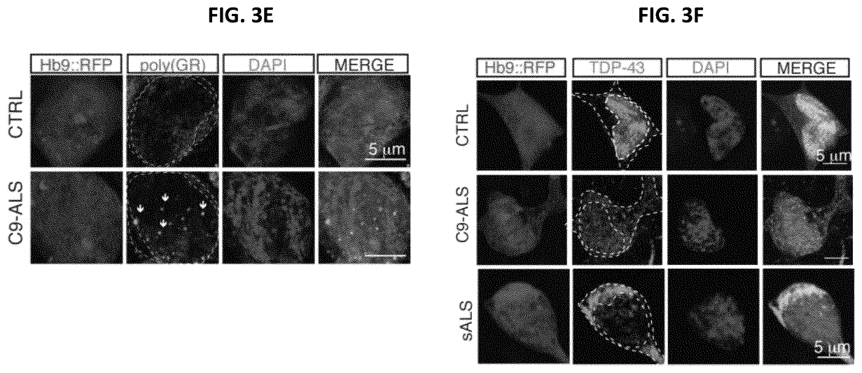

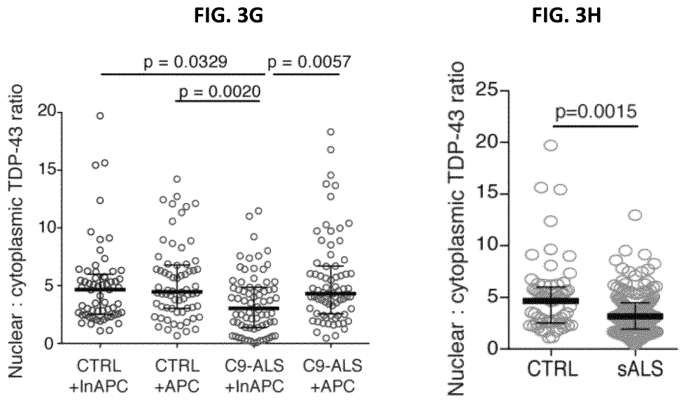

These generated iMNs display the typical spinal cord motor neuron morphology (FIG. 2A), gene expression (FIG. 2G-2H), and electrophysiology signature (FIG. 2D-2F), similar to mature human motor neurons. In particular, these iMNs generated from ALS patient cells recapitulate the actual ALS motor neuron degenerative phenotype, i.e. ALS patient motor neurons are more likely to break down and die than healthy control motor neurons (FIG. 3B, 3C).

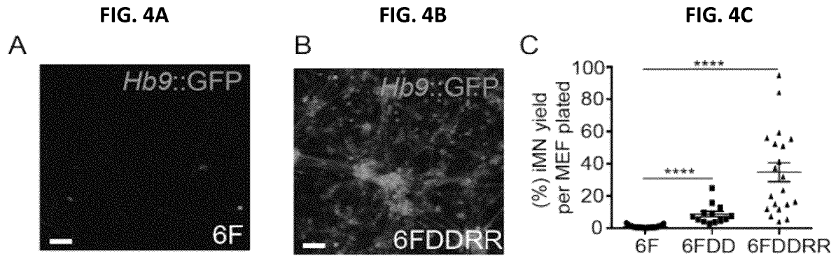

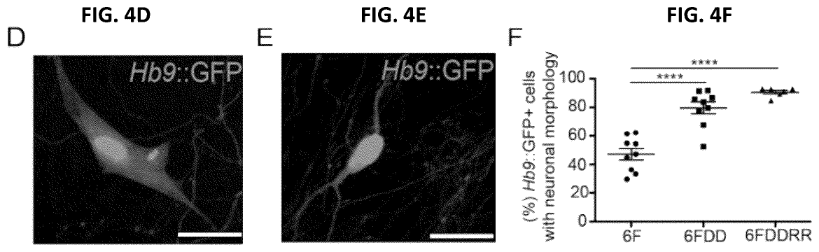

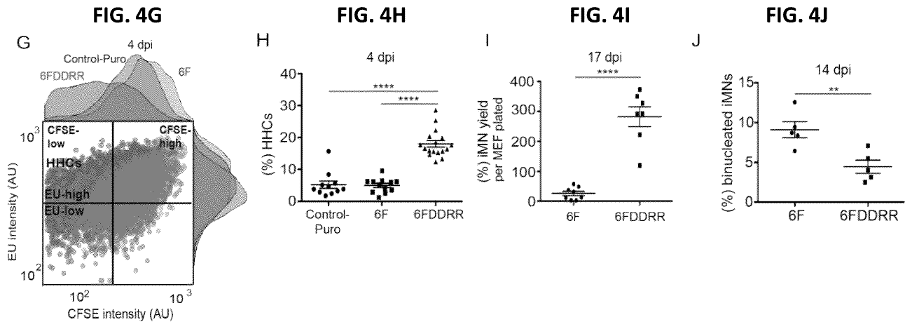

Another approach to generating motor neurons is transcription factor mediated reprogramming of fibroblasts into induced motor neurons (fib-MNs). Starting with patient fibroblasts rather than iPSCs can reduce timelines by several weeks. By adding DDRR (p53DD (DD), a p53 mutant lacking a DNA-binding domain, RepSox, a TGF-beta inhibitor 27, and a Ras mutant (hRasG12V)) to the 7 factor protocol described above, reprogramming was increased significantly (FIG. 4A-C). FIG. 4 shows how a specific chemical and genetic cocktail enables near-deterministic reprogramming of fibroblasts into motor neurons. This combination, which has been named DDRR, endows cells with the ability to mitigate an inherent antagonism between transcription and DNA replication caused by a build-up of DNA supercoiling induced by both processes. This increases the population of hypertranscribing and hyperproliferating cells (FIG. 4G, H) (HHCs), and these cells reprogram with near-deterministic efficiency (FIG. 4H). This results in much higher iMN yields with reduced heterogeneity (FIG. 4F, 4I). This data shows results in mouse cells, but human iMNs reprogramed using this enhanced DDRR protocol similarly have enhanced yields and reduced occurrence of partially reprogrammed cells (FIG. 4I, 4J). In one embodiment, the DDRR method is used to generate the neurons (e.g., motor neurons).

### Testing Prospective Treatments

Prospective treatments include, but are not limited to, administration of one or more pharmaceutically active agents, such as small interfering RNAs (siRNAs), antisense oligonucleotides (ASOs), cholinesterase inhibitors (e.g., donepezil, rivastigmine, and galantamine), NMDA receptor antagonists (such as memantine or dextromethorphan hydrobromide), antiglutamatergic agents (such as riluzole), edaravone, CYP450 2D6 inhibitors (such as quinidine sulfate), SOD1 gene or expression inhibitors (such as tofersen sodium, AVXS-301, NI-204.12G7, and VY-SOD101), TGF-beta type II receptor modulators (such as NVP-13), C9orf72 gene modulators (such as WVE-3972-01, C9orf72 ZFP-TF gene therapy, and C9orf72 gene or expression inhibitors such as NI-308 and IONIS-BIB5Rx), GRIA1 gene modulators (such as LSP-GR1), cytosolic phospholipase A2 inhibitors (including ASOs to cytosolic phospholipase A2), GRIA3 gene modulators (such as GluR3 splice modulating oligomers), TAR DNA binding protein 43 modulator (such as an anti-TDP-43 monoclonal antibody), CT-814, masitinib mesylate, YYB-103, mecobalamin, mesenchymal stem cells (such as NTF-SC), a stem cell therapy (such as HYNR-CS), a heat shock protein 70 (Hsp70) inducer (such as arimoclomol citrate), adrenocorticotropic hormone, alirinetide, deferiprone, EPI-589, TRPA1 agonist (such as FLX-787), TRPV1 agonist, human albumin, leukotriene CysLT1 (LTD4) receptor antagonist (such as ibudilast), calcium sensitizers (such as levosimendan), NP-001, NSI-566RSC, phenylbutyric acid sodium/tauroursodeoxycholic acid, viral entry inhibitors (such as RPI-MN), Cu(II)-ATSM, donaperminogene seltoplasmid, dihydrofolate reductase inhibitors (such as pyrimethamine), antiamyloidogenic agents (such as AAD-2004), GABA (B) receptor agonists, baclofen, acamprosate, CNS 10-NPC-GDNF, aldo-keto reductase family 1 member B10 (AKR1B10) inhibitors (such as caffeic acid phenethyl ester), acetylcholine release enhancers (such as cutamesine hydrochloride), necroptosis inhibitors (such as DNL-747 and DNL-788), mitogen-activated protein kinase kinase kinase 12 (MAP3K12) inhibitors (such as GDC-0134), anti-CD14 antibodies, calcium channel modulators (such as RNS-60), WJ-MSC, 748940, AMPA receptor antagonists (such as perampanel), WN-1316, Q-cells, AAV9-mycUPF-1, AGT-110, anti-CD154 (anti-CD40L) antibodies (such as AT-1501 and 999737), AX-200, NF-kappaB modulators (such as CAT-4001), C5a anaphylatoxin chemotactic receptor 1 (C5aR) antagonists (such as HC-[OP (D-Cha) WR]), HTL-14242, IMS-088, IZ-10023, exportin-1 receptor antagonists (such as KPT-350), glutamate ionotropic antagonists (such as LSP-GR1 and LSP-GR3), MIR155 inhibitors (such as MRG-107), NNZ-4945, antimitotic drugs (such as noscapine), nicotinamide phosphoribosyltransferase activators (such as P7C3), PRCN-829, regenemab, SYN-1, melanocortin receptor modulators (such as sephin-1), TW-002, U-Cord-Cell, angiogenesis inhibitors (such as WTX-101), rhCDNF, 864757, 928217, cyclic AMP, levodopa (L-dopa), cabidopa, ropinirole, pramipexole, rotigotine, amantadine, trihexyphenidyl, benztropine, selegiline, bromocriptine, tolcapone, pergolide, ropinirole, phenylzine, tranylcypromine, isocarboxazid, entacapone, artane, an antidepressant, rilutek, radicava, a dopamine agonist, a MAO-B inhibitor (such as rasagiline or rasagiline mesylate), recombinant hepatocyte growth factor, fast skeletal muscle troponin activators (such as rasagiline), GABA aminotransferase inhibitors (such as retigabine), anti-interleukin-6 receptor subunit (such as tocilizumab), catechol-O-methyltransferase inhibitors, anticholinergic agents, spinraza, tetrabenazine, antipsychotic agents, levetiracetam, clonazepam, antipsychotic agents, mood-stabilizing agents, and amantadine. A prospective treatment can be with a single pharmaceutically active agent or a combination of two or more pharmaceutically active agents. These treatments may be administered by the oral, intravenous, parenteral, ophthalmic, nasal, pulmonary, and topical route.

In one embodiment, the prospective treatments tested include two or more pharmaceutically active agents that have different mechanisms of action. In another embodiment, the prospective treatments tested include a library of pharmaceutically active agents, for instance, that include ten or more (e.g., twenty or more) pharmaceutically active agents where each has a different mechanism of action.

The treatments may be tested against the generated neurons by applying the prospective treatment. In one embodiment, the neurons are placed in a plurality of wells of a microarray and a prospective treatment is applied to each microarray. In this manner, a given prospective treatment can be tested in replicate (for instance, a prospective treatment can be tested against two, three, or four samples of the neurons).

### Assessing Neurons Following Treatment

In one embodiment, the neurons are assessed by single cell longitudinal tracking (i.e., particular cells are tracked on an individual basis over time, such as by imaging techniques).

In one embodiment, the neurons are assessed following treatment with an assay (e.g., an assay that takes a measurement at only one time point), for example, a protein localization assay (e.g., to determine the protein localization of TDP-43, DPR protein aggregates, Ran, RanGAP1, TDP43, LmnB, TPNO3, KPNA3, XPO5) and RNA by IF, FISH. Protein functional activities correspond with their subcellular expression and molecular complexing interactions. Protein localization can be effectively demonstrated with fluorescence microscopy based techniques or fractionation procedures. A broad spectrum of fluorescence imaging can be accomplished by using recombinant reporter proteins, fluorescent dyes, or fluorophore labeled molecules (i.e. protein specific antibodies).

In one embodiment, neurons are assessed by single neuron longitudinal tracking (e.g., by automated microscopy). Individual neurons are observed over a period of time and assessed at certain time points to be determine whether each neuron continues to subsist. Longitudinal tracking refers to the ability to track individual neurons over time.

Another survival assay, a colorimetric assay, involves the measurement of a biochemical marker to evaluate metabolic activity of the cells. Reagents used in colorimetric assays develop a color in response to the viability of cells, allowing the colorimetric measurement of cell viability via a spectrophotometer. Colorimetric assays are applicable for adherent or suspended cell lines, easy to perform, and comparably economical. Commercial kits of colorimetric assays are available.

Yet another survival assay, fluorometric assays of cell viability and cytotoxicity, are easy to perform with the use of a fluorescence microscope, fluorometer, fluorescence microplate reader or flow cytometer, and they offer many advantages over traditional dye exclusion and colorimetric assays. Fluorometric assays are also applicable for adherent or suspended cell lines and easy to use. These assays are more sensitive than colorimetric assays. Commercial kits of fluorometric assays are available.

Luminometric assays provide fast and simple determination of cell proliferation and cytotoxicity in mammalian cells. These assays can be performed in a convenient 96-well and 384-well microtiter plate format and detection by a luminometric microplate reader. A useful feature of the luminometric assays is the persistent and stable glow-type signal produced after reagent addition. This attribute can be harnessed to produce both viability and cytotoxicity values from the same well. Commercial kits of luminometric assays are available.

The assessment can be performed by various cell imaging techniques, such as ion imaging (e.g., observing changes in ion concentrations); methods for quantifying protein-protein interactions such as Förster resonance energy transfer (FRET) and bioluminescence resonance energy transfer (BRET); monitoring protein and vesicle trafficking such as fluorescence recovery after photo-bleaching (FRAP); observing processes close to the cell membrane, such as total internal reflection (TIRF); monitoring gene expression and/or protein transport such as by photoactivation; investigating processes in depth such as with multiphoton excitation (MPE) microscopy; studying cellular dynamics at nanometer scale such as with stimulated emission depletion (STED) microscopy; spatial measurements in living cells, such as with fluorescent lifetime imaging (FLIM); and label-free methods using vibrational contrast such as coherent anti-Stokes raman scattering (CARS) microscopy and stimulated Raman scattering (SRS) microscopy.

Ion imaging (such as for calcium, chloride, magnesium) can be performed using either fluorescent dyes or proteins especially designed to change their emission behavior upon calcium binding. The ion composition in the cytosol of cells determines many crucial functions of cells such as excitability of neurons, gene transcription and cell movement. Also, the imaging of intracellular pH-levels or voltage is possible with special fluorescent dyes. A special technique for imaging changes in ion levels, pH levels or voltage are ratiometric imaging methods. These allow exact determination of, for instance, the intracellular calcium concentration instead of monitoring relative changes as it is done in non-ratiometric methods.

To detect dynamic protein interactions, FRET (Förster resonance energy transfer) and BRET (bioluminescence resonance energy transfer) events can be imaged in live-cell experiments. FRET is a useful tool for quantifying molecular dynamics, such as protein-protein interactions, protein-DNA interactions, and protein conformational changes. FRET imaging normally uses derivatives of GFP (green fluorescent proteins), particularly CFP and YFP (cyan and yellow fluorescent protein, respectively), which are each attached to proteins of interest using molecular biology methods. Then the CFP molecule is excited with fluorescent light. As soon as the proteins of interest are in close spatial proximity (<20 nm) the CFP will act as a donor and transfer the energy which is emitted in the form of light to the YFP, which acts as an acceptor. A shift from blue fluorescence emitted from the CFP to yellow fluorescence emitted from the YFP is then observed. In the case of BRET the donor is a bioluminescent molecule (e.g. luciferase derivatives) that acts as the donor and, like in FRET, GFP derivatives act as acceptor.

A method often applied for monitoring protein or vesicle trafficking is fluorescence recovery after photo-bleaching (FRAP). Here a fluorescent protein (usually GFP) is attached to a protein of interest (i.e. a protein whose movement is to be monitored). Usually, the whole cell is initially fluorescent as the protein might be abundant in the whole cell. Then a certain region of the cell, often cell processes like axons or dendrites in neuronal cells, is exposed to high intensities of light (usually laser light) and the fluorescence in that particular region is destroyed (bleached). As the protein of interest is moving, proteins from other parts of the cell will reinvade the bleached region at a certain speed and the fluorescence in the bleached region recovers. This provides insight into intracellular transport dynamics.

A technique for observing events that are located in or close to the plasma membrane of a cell is TIRF (total internal reflection) microscopy. By using an evanescent field for fluorochrome excitation which only penetrates the cell 60-250 nm, TIRF microscopy enables the imaging of events occurring in or close to the plasma membrane (e.g. molecule transport to the plasma membrane) without being outshone by fluorescence from molecules inside the cell.

Yet another method called photoactivation selectively labels certain regions or areas of interest within a cell or a whole organism. For photoactivation especially designed dyes or fluorescent proteins such as photoactivatable green fluorescent protein (paGFP) or Kaede are used. These fluorophores are not fluorescent in their normal state. However, after illumination with light of certain wavelengths, these fluorophores can be activated to fluoresce like conventional fluorophores. In many cases, these proteins are genetically fused to certain proteins of interest whose expression or transport can then be monitored. Methods such as FRAP or particle tracking can then be applied to further investigate the protein of interest.

Multiphoton excitation (MPE) microscopy enables deeper penetration into tissue, because the near-infrared excitation light, having a longer wavelength, is less scattered compared to the short-wavelength light used for single-photon excitation. The non-linear nature of the MPE technique restricts photobleaching and phototoxicity to the area in the focus.

Stimulated emission depletion (STED) microscopy allows investigation of structures beyond the optical resolution limit. This technique uses the property of fluorescent dyes, being stimulated to emission, to eliminate detectable signals. Intracellular structures down to 50-70 nm, such as small intracellular structures, can be imaged. The stochastic independence of STED enables very fast imaging compared to other super-resolution techniques. Video rate STED-acquisition has been achieved, which allows cellular dynamics to be studied in real time.

Fluorescent lifetime imaging has the advantage that the data is not dependent on the intensity of the signal. Therefore it is not influenced by common artifacts like photobleaching and concentration variation. Using time-correlated single photon counting, FLIM images can be reconstructed from data of single molecule detection. The minimal change of the fluorescence lifetime in sub-nanoseconds can be registered and analyzed. The method is versatile for investigation of various kinds of extra- and intracellular environmental modification which lead to an alteration of the fluorescence lifetime. FLIM-based FRET analysis is insensitive to the intensity of the emission and thereby increases the precision of the quantitative data.

Coherent anti-Stokes Raman Scattering (CARS) microscopy and stimulated Raman Scattering (SRS) microscopy are non-linear confocal methods which are not dependent on fluorescent dyes. These label-free methods image the vibration states of specific chemical bonds in the sample. The accumulation of specific chemical bonds in living organisms, such as the lipid in myelin around the axons, can be imaged in high resolution and excellent signal-to-noise quality, without any need for staining.

### Selection of Patients for Clinical Trials

Another embodiment is a method for selecting patients for a clinical trial involving a prospective treatment for a neurodegenerative disorder. The method involves for each patient (i) generating neurons from fibroblasts or from induced pluripotent stem cells which are generated from somatic cells obtained from the patient; (ii) testing the prospective treatment against the generated neurons; (iii) assessing the neurons following treatment; and (iv) selecting patients for the clinical trial based on the results of the assessment in step (iii). In one embodiment, patients are selected based on an enhanced survival rate of the generated neurons after the prospective treatment compared to prior.

### Examples of Methods of Identification of Suitable Treatments

In one embodiment, the process for generating neurons and assessing prospective treatments includes obtaining a skin biopsy, culturing fibroblasts, and reprogramming the fibroblasts, as described above, using the 7 factor protocol. The 7 factor protocol results in the generation of induced motor neurons (iMN). Those generated iMN can then be quantified before and after a prospective treatment using either a survival assay or a one-and-done assay.

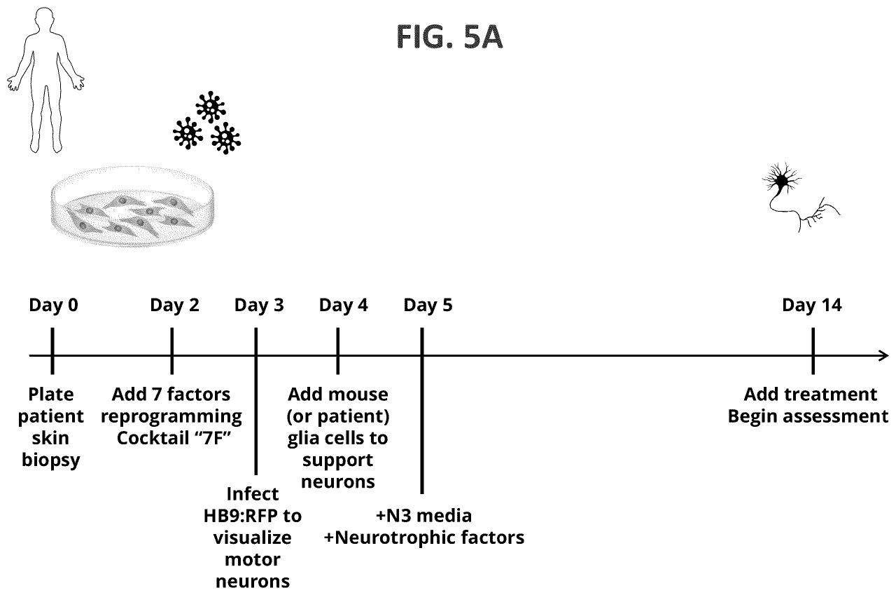

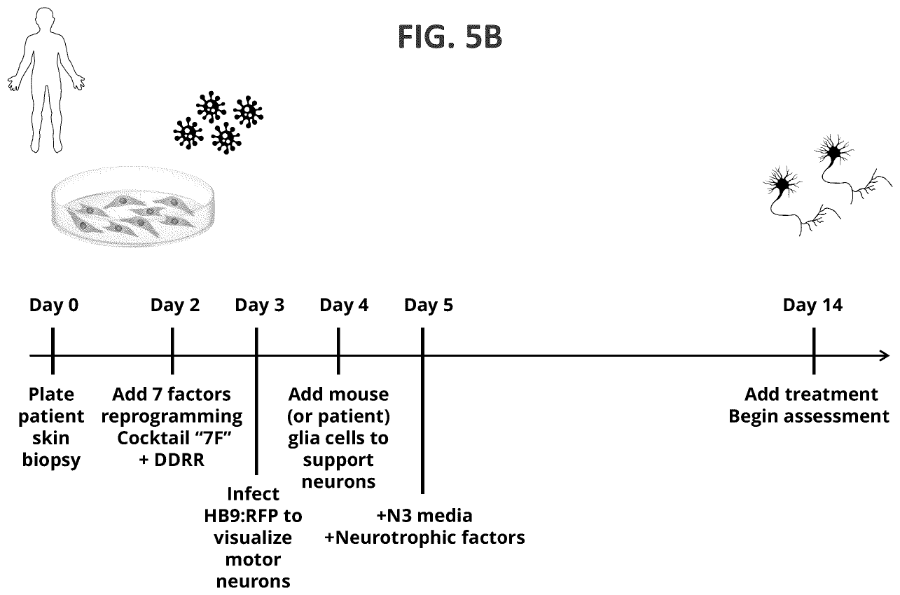

FIG. 5A shows that exemplary method. The method commences at Day 0, where a skin biopsy is performed on a patient and then plated on media. At Day 2, the 7F reprogramming cocktail is introduced to initiate generation of neurons. At Day 3, the media is infected with the HB9: RFP plasmid to visualize the motor neurons. At Day 4, mouse or patient glia cells are added to the media to support the generated neurons. At Day 5, N3 media and neurotrophic factors are added. At Day 14, the motor neurons are ready for the addition of treatment and assessment of its efficacy.

Another process for reprogramming cells in accordance with an alternate embodiment of the present invention involves taking a skin biopsy, culturing fibroblasts, and reprogramming the fibroblasts, as described above, but using the 7 factor protocol along with DDRR to generate iMN. Those generated iMN can then be quantified using a survival assay.

FIG. 5B shows that method in greater detail. The method commences at Day 0, where a skin biopsy is performed on a patient and then plated on media. At Day 2, the 7F reprogramming cocktail and DDRR are introduced to initiate generation of neurons. At Day 3, the media is infected with the HB9: RFP plasmid to visualize the motor neurons. At Day 4, mouse or patient glia cells are added to the media to support the generated neurons. At Day 5, N3 media and neurotrophic factors are added. Once more, at Day 14, the motor neurons are ready for the addition of treatment and assessment of its efficacy.

Motor neurons generated from ALS patients have more neurodegeneration as compared to healthy controls (FIG. 3A-3C). Degeneration can be assessed by automated microscopy, imaged daily over for instance two weeks, and single neuron tracking of the HB9::RFP positive cells in the microscopy image series. This can be performed in 96-well plates where each well is a composite tiling (4×4 at 4× objective) covering the entire well so that all neurons are tracked. Additionally, motor neurons from ALS patients have localization defects in TDP-43 protein, which is known to form aggregates in 97% of patients, and form dipeptide repeat proteins aggregates in motor neurons from C9ORF72-ALS patients and these aggregates are also seen post mortem in the brains of C9ORF72-ALS patients (FIG. 3D-3H). Patient motor neurons samples generated through this methodology recapitulate gene expression changes observed in ALS patient post-mortem neurons from multiple studies (FIG. 2G-2H).

Tests of drugs tested in ALS clinical trials on ALS patients show the promise of this approach (FIG. 3B). Drugs that have done best in clinical trials, such as the two FDA approved drugs riluzole and edaravone, do best in the patient motor neurons, and drugs that failed in clinical trials do worse. However, no drugs are toxic (which would be an hR value>1) as would be expected for drugs in a clinical setting. Importantly, pharmacologic interventions that reduce cytoplasmic TDP-43 mislocalization can rescue C9ORF72 and sporadic ALS iMN survival in the longitudinal tracking assay.

TAR DNA binding protein 43 (TDP-43) is a versatile RNA/DNA binding protein involved in RNA-related metabolism. TDP-43 is a highly conserved and ubiquitously expressed nuclear protein with roles in transcription and splicing regulation. Hyper-phosphorylated and ubiquitinated TDP-43 deposits act as inclusion bodies in the brain and spinal cord of patients with the motor neuron diseases: amyotrophic lateral sclerosis (ALS) and frontotemporal lobar degeneration (FTLD). While the majority of ALS cases (90-95%) are sporadic (sALS), among familial ALS cases 5-10% involve the inheritance of mutations in the TARDBP gene and the remaining (90-95%) are due to mutations in other genes such as: C9ORF72, SOD1, FUS, and NEK1. The majority of sporadic ALS patients (up to 97%) also contain the TDP-43 protein deposited in the neuronal inclusions, which is suggestive of its role in the ALS pathology.

The methods can be used to predict efficacy in the clinic by testing on patient neurons to confirm restoration of TDP-43 protein localization (localization in WT and treated neurons should be in the nucleus and untreated patients would see more protein in the cytoplasm), and recovery of motor neuron survival to levels similar to healthy controls. In one embodiment, fibroblasts obtained from the patient are used to generate motor neurons (for example, within 2, 3 or 4 weeks) and then assessed by a TDP-43 localization assay (for example, taking 1 week), which is fast enough to support clinical trial enrollment or to provide treatment information to clinicians.

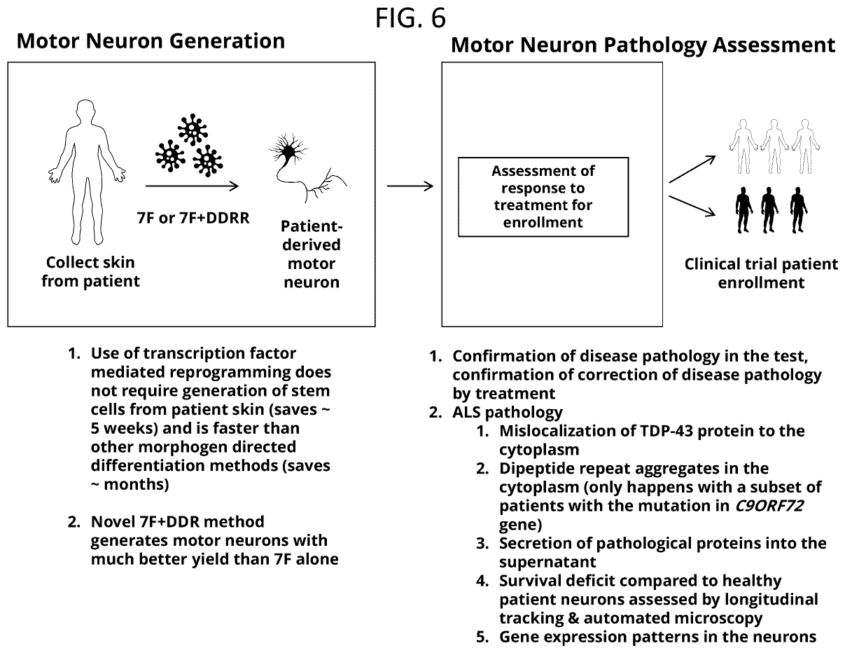

FIG. 6 shows a summary of motor neuron generation and motor neuron pathology assessment, in accordance with an embodiment of the invention. In the motor neuron generation phase, described above, use of transcription factor mediated reprogramming does not require generation of stem cells from patient skin (which can save several weeks) and is faster than other morphogen directed differentiation methods (which can save months). In one embodiment, the novel 7F+DDR method is used to generate motor neurons and produces them in better yield than 7F alone. At the assessment step, there is confirmation of disease pathology in a test and confirmation of correction of disease pathology by treatment.

For the exemplary ALS pathology, one or more of the following analyses can be performed: (1) Mislocalization of TDP-43 protein to the cytoplasm; (2) Dipeptide repeat aggregates in the cytoplasm (only happens with a subset of patients with the mutation in C9ORF72 gene); (3) Secretion of pathological proteins into the supernatant; (5) Survival deficit compared to healthy patient neurons assessed by longitudinal tracking & automated microscopy; and (5) Gene expression patterns in the neurons.

In addition to testing dose-response where response is a well-established efficacy readout such as survival or protein localization, biomarker exposure-response can also be modeled where the response is a putative biomarker that can be translated from human neurons in vitro to an animal model in vivo and then back to humans in a clinical trial. Therefore the readout of the in vitro testing assay could also be a biomarker/pharmacodynamic (PD)-like readout such as enhanced levels of exocytosis, enhanced levels of TDP-43 or DPR secretion.

In this protocol the neurons are created as described above and the treatment is administered to the cell culture in a single dose, or in multiple ascending doses to elicit a dose response.

One embodiment of this method for ALS patients is described below. First, it is verified that iMNs from the C9ORF72 and sporadic ALS patient lines display significantly faster iMN degeneration and a lower nuclear:cytoplasmic TDP-43 ratio than controls. All phenotyping assays in this study are performed in biological triplicate with Hb9::RFP-labelled iMNs. Then, each ALS line is compared to 25 control lines and require the hazard ratio of the patient iMNs (likelihood of death) to be higher than the composite of the controls with a p-value<0.05 by log-rank test.

TDP-43 localization is detected using total TDP-43 immunocytochemistry to check if the disease lines exhibit a significantly lower nuclear:cytoplasmic TDP-43 ratio in iMNs compared to the average of the control lines (One-way ANOVA). For C9ORF72 ALS lines, immunocytochemistry is used to verify that C9ORF72 iMNs possess a significantly greater number of poly (GR)+ DPR aggregates than the average of the control lines (One-way ANOVA).

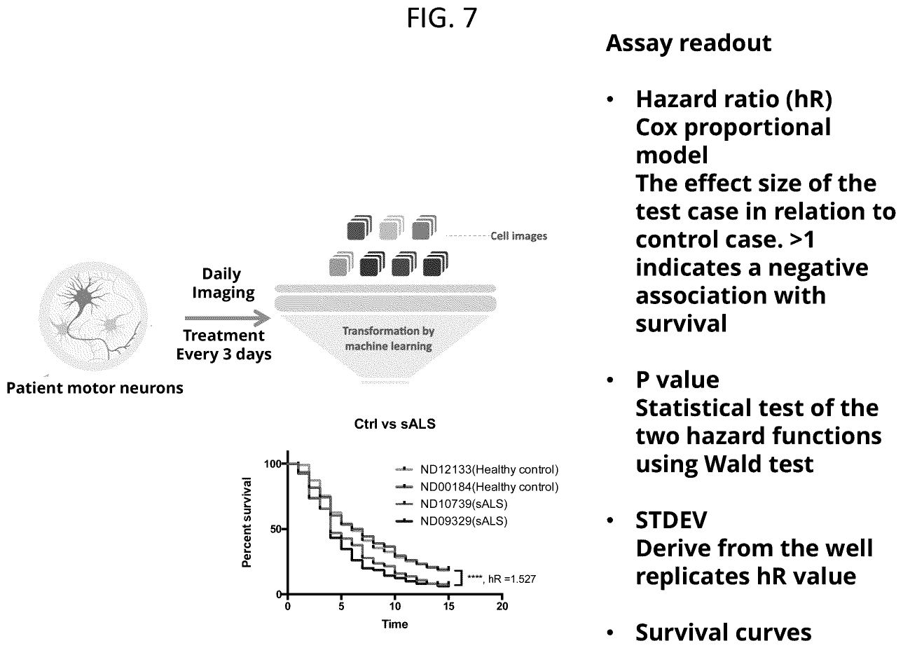

As shown in FIG. 7, an iPSC 7F iMNs survival assay was established and image analysis technologies determined the neuron survival rate. Daily imaging was performed to collect data at multiple time points over ˜2 weeks, and then analysis of the data enabled single cell longitudinal tracking during the progressive stages of cell death. The hazard ratio (hR) derived from the Cox model is used to indicate the survival rate of neurons, where a ratio greater than 1 indicates a negative association with survival. A P value is also calculated, which functions as a statistical test of the two hazard functions using the Wald test. In addition, a standard deviation, which replicates the hR value, and survival curves are calculated and graphed.

Conversion of iPSCs into induced motor neurons: Reprogramming was performed in 96-well plates that were sequentially coated with gelatin (0.1%, 1 h at 37° C.) and laminin (overnight at 4° C.). iPSC-derived secondary fibroblasts for at least 48 h and either used directly for retroviral transduction or passaged before transduction for each experiment. Seven iMN were added in fibroblast medium with 5 μg/ml polybrene. For iMNs, cultures were transduced with lentivirus encoding the Hb9::RFP reporter 24 h after transduction with transcription factor-encoding retroviruses. On day 5, primary mouse cortical glial cells from P2-P4 ICR pups (male and female) were added to the transduced cultures in glia medium containing MEM (Life Technologies/ThermoFisher Scientific of Waltham, MA), 10% donor equine serum (HyClone/GE Healthcare Life Sciences of Pittsburgh, PA), 20% glucose (Sigma-Aldrich of St. Louis, MO), and 1% penicillin/streptomycin. On day 6, cultures were switched to N3 medium containing DMEM/F12 (Life Technologies), 2% FBS, 1% penicillin/streptomycin, N2 and B27 supplements (Life Technologies), 7.5 M RepSox (Selleck Chemicals of Houston, TX), and 10 ng/ml each of GDNF, BDNF, FGF, and CNTF (R&D Systems of Minneapolis, MN). The iMN cultures were maintained in N3 medium, changed every other day.

Induced neuron survival assay: Hb9::RFP⁺iMNs appeared between days 13-16 after retroviral transduction. RepSox, GDNF, BDNF, FGF, and CNTF) were removed at day 17 and the survival assay was initiated. Longitudinal tracking was performed by imaging neuronal cultures in Molecular Devices ImageExpress once every 24 h starting at day 17. Tracking of neuronal survival was performed using image analysis pipeline. Neurons were scored as dead when their soma was no longer detectable by RFP fluorescence. All neuron survival assays were performed at least twice, with equal numbers of neurons from three individual replicates from one of the trials being used for the quantification shown. All trials quantified were representative of other trials of the same experiment.

Automated longitudinal single cell tracking and survival characterization: Prior to fully automated processing, 4 images covering a single well of a 96 well plate are stitched to create a single 2×2 composite images covering the entire well. This step is automated for all wells and plates in the run. After stitching, the automated processing steps include 1) soma detection; 2) longitudinal soma tracking and 3) statistical modeling. Step 1 is performed as the images are acquired (after stitching) and reserving only the tracking and statistical characterization for the end after the complete time series is acquired—greatly improving system throughput as compared to processing all images at the end of the experiment.

Soma detection is performed using a mathematical morphology method library (Legland, D., Arganda-Carreras, I. & Andrey, P. MorphoLibJ: integrated library and plugins for mathematical morphology with ImageJ. Bioinformatics 32, 3532-3534 (2016)). One challenge in soma detection is discriminating soma from complex cellular arbors and a small number of fluorescently labeled non-neuronal cells in the culture. The inventors model motor neuron soma as having a Gaussian intensity distribution with a relatively steep gradient from the peak intensity. This is accomplished (after pre-processing the images with a normalization, background removal and bandpass filter to remove slender neuronal processes) by a peak detection method followed by flooding down conditioned by the distance from the peak and the cell body. Soma detection candidates are further conditioned by selecting the best soma candidate if multiple candidates are detected on a single cell body and by size. Neuronal processes are then detected using an algorithm built from the MorpholibJ libraries and an optimized Java implementation of the ImageJ function Analyze Branches (Arganda-Carreras, I., Fernández-González, R., Muñoz-Barrutia, A. & Ortiz-De-Solorzano, C. 3D reconstruction of histological sections: Application to mammary gland tissue. Microsc. Res. Tech. 73, 1019-1029 (2010)). Soma are then classified using a neuron classification model, and non-neuronal soma are removed from the detection for the initiation of tracking.

Longitudinal soma tracking is performed after all the time series images are acquired and soma detection is performed for all wells. Single cell soma tracking is automated on the pipeline using the Lineage Mapper algorithm developed by the National Institute of Standards and Technology (Chalfoun, J. et al. Lineage mapper: A versatile cell and particle tracker. Sci. Rep. 6,36984 (2016)). The output is x, y, t data for each soma. Single cell longitudinal survival summary data and survival function (and plotted curves) are then aggregated from the tracking output. Only soma which have been classified as neurons and exist in the first frame are considered for analysis (neurons which appear in later frames are ignored).

Statistical analysis. Differences in survival between test and control conditions are then assessed using an implementation of the Cox Proportional Hazards model (Cox, D. R. Regression Models and Life-Tables. J. R. Stat. Soc. Series B Stat. Methodol. 34, 187-220 (1972)). The model generates the hazard ratio (hR) and p values indicating whether survival differences between conditions are significant. The hazard ratio is used as the assay metric. It is the ratio of the hazard rates, the risk of cell death at that moment given that the cell has survived until that time point, for the each of the test and control conditions. For example a toxic compound that kills cells at twice the rate of DMSO would have a ratio of 2. Therefore a test condition with a hazard ratio less than 1 indicates that the condition enhances motor neuron survival as compared to the control.

3 PIKFYVE inhibitors were profiled as lead compounds in a dose response in biological triplicate across the iMN panel for TDP-43 localization, C9ORF72 DPR aggregates (control and C9ORF72 only), and iMN survival to identify a suitable treatment. The fraction of C9ORF72 and sporadic ALS lines that are significantly rescued in both the proteostasis and survival assays by PIKFYVE inhibition were determined. A significant reduction in poly(GR) levels for C9ORF72 lines demonstrated successful treatment. A therapeutic window was also determined to see if it varies across ALS patient iMNs. The data indicates that high, micromolar doses of PIKFYVE inhibitors do not cause ALS iMN toxicity.

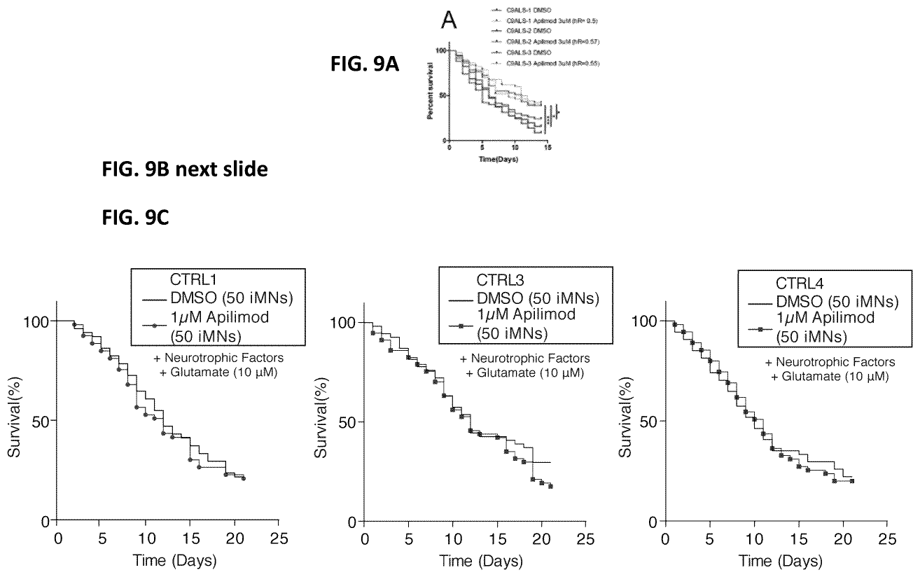

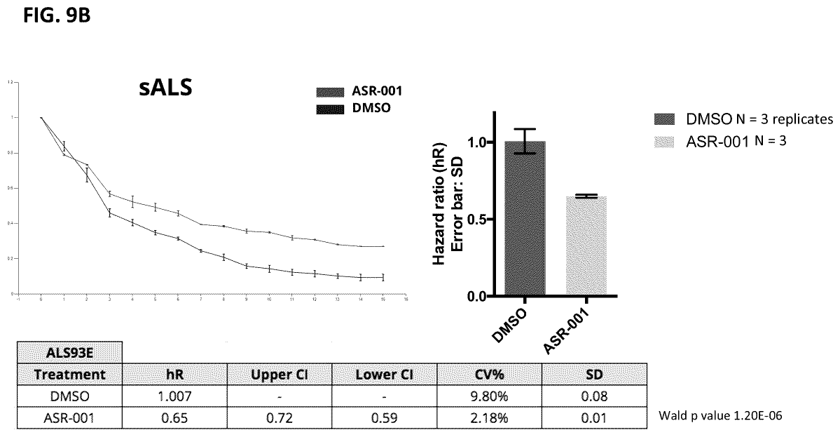

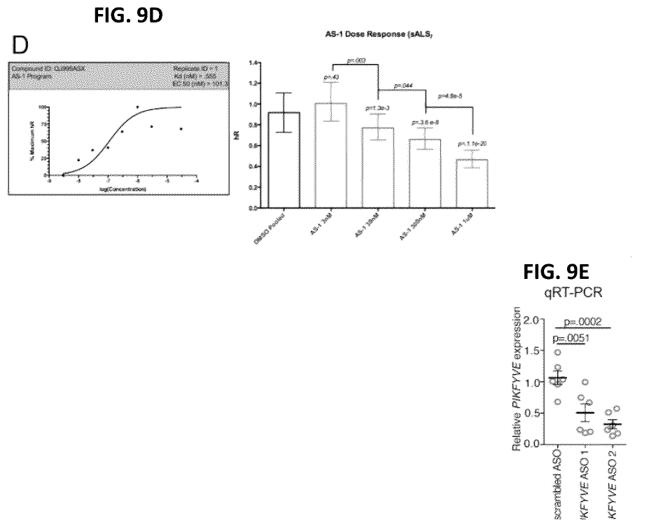

Of the hit compounds that were efficacious against most sporadic ALS patient iMNs, the PIKFYVE inhibitor apilimod was among the most potent (FIG. 9A, 9B). PIKFYVE inhibition rescued ALS iMN survival in a dose-dependent manner (FIG. 9D) and did not affect control iMN survival (FIG. 9C). Antisense oligonucleotide (ASO) knock down of PIKFYVE also rescued ALS iMN survival (FIG. 9E).

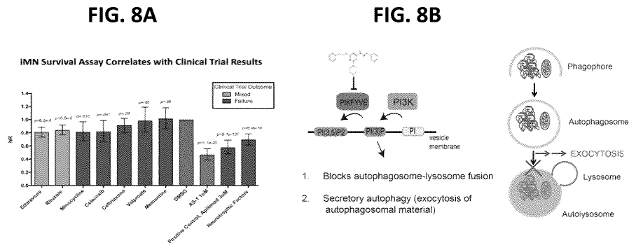

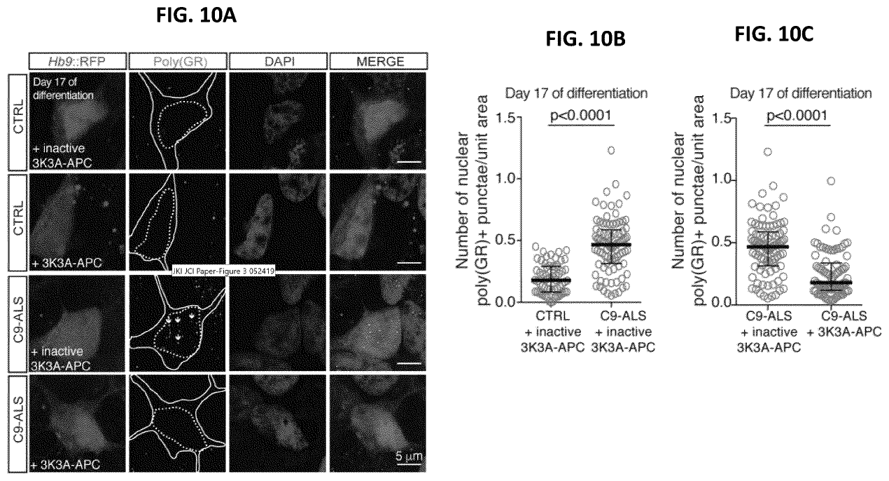

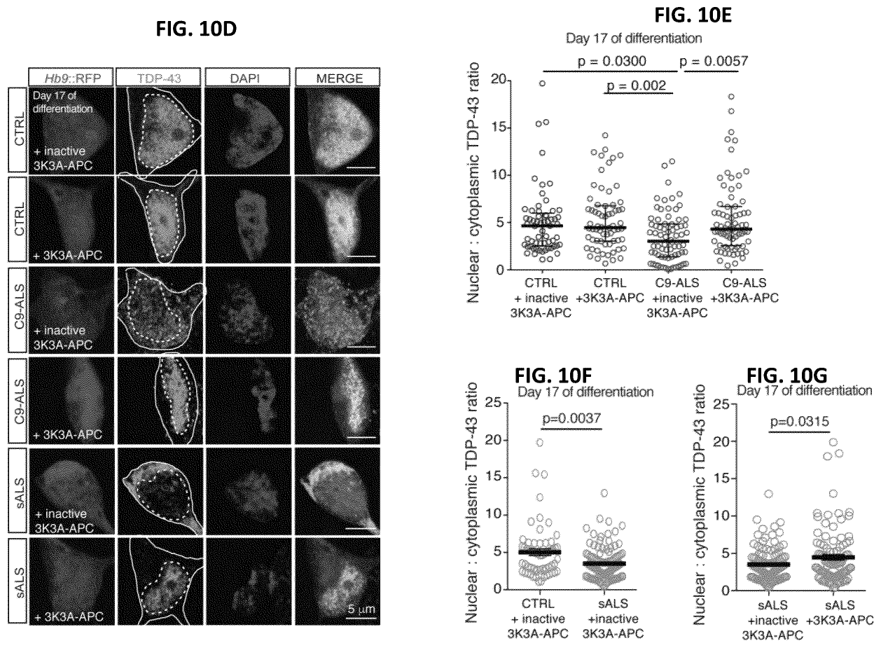

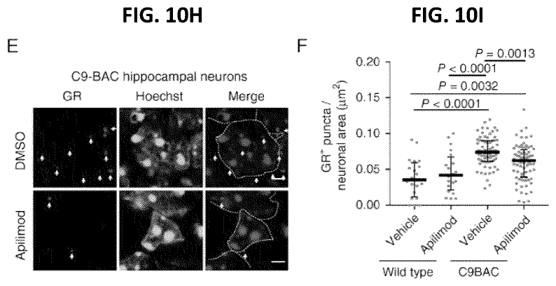

PIKFYVE is a Class-III Phosphatidylinositol-5-kinase that synthesizes PI(3,5)P2 from PI3P. PI3P is used for autophagosome formation and engulfment of proteins designated for degradation, but its conversion to PI(3,5)P2 facilitates autophagosome-lysosome fusion. PIKFYVE inhibition therefore impairs the fusion of lysosomes with multivesicular bodies and autophagosomes, and neural cells respond by secreting the content of these organelles to maintain cellular proteostasis (FIG. 8B). ALS patient iMNs exocytose C9ORF72 DPRs and TDP-43 upon PIKFYVE inhibition (FIG. 8B). This reduces cytoplasmic TDP-43 in C9ORF72 and sporadic ALS iMNs and rescues iMN survival (FIG. 9A, 9B). Small molecule PIKFYVE inhibition also reduces DPR levels in C9ORF72 BAC transgenic mice (FIG. 10H, 10I) and protects against glutamate-induced excitotoxicity in C9ORF72+/− mice by lowering glutamate receptor levels, indicating that it restores proteostasis in vivo. Thus, the therapeutic mechanism of PIKFYVE inhibition is to induce secretory autophagy which restores neuronal proteostasis. The secreted materials can also serve as target engagement biomarkers.

A TDP-43 localization assay was established in fib-7FDDRR iMNs as a reliable patient screening tool. Skin fibroblasts were obtained from 20 control and 20 sporadic ALS patients. iPSCs were then generated and the drug response was measured in iPSC-7F iMNs and fib-7FDDRR iMNs for the TDP-43 localization assay to identify a correlation between disease phenotypes in iPSC 7F and fib-7FDDRR iMNs.

Treatment of iMNs with the test compounds induces secretory autography in ALS patient motor neurons. The method commences with the addition of the test compound to ALS patient motor neurons in media. Following addition of the test compound, supernatant is collected from the media and exosomes are purified. Western blot and FACS analysis are then used to determine the efficacy of the treatment.

The foregoing description and drawings should be considered as illustrative only of the principles of the invention. The invention is not intended to be limited by the preferred embodiment and may be implemented in a variety of ways that will be clear to one of ordinary skill in the art. Numerous applications of the invention will readily occur to those skilled in the art. Therefore, it is not desired to limit the invention to the specific examples disclosed or the exact construction and operation shown and described. Rather, all suitable modifications and equivalents may be resorted to, falling within the scope of the invention. All references cited herein are incorporated by reference.

1. A method for identifying a suitable treatment for a patient having a neurodegenerative disorder comprising:
(i) generating motor neurons from (i) induced pluripotent stem cells from somatic cells or (ii) fibroblasts obtained from a patient;
(ii) testing two or more prospective treatments against separate groups of the generated motor neurons; and
(iii) assessing the motor neurons following each treatment.

2. The method of claim 1, wherein the motor neurons are generated by morphogen directed differentiation, linear conversion, or any combination of any of the foregoing.

3. The method of claim 1, wherein the fibroblasts are reprogrammed with at least one transcription factor and optionally a TGF-beta inhibitor, a Ras mutant, a p53 mutant lacking a DNA-binding domain, or any combination of any of the foregoing.

4. The method of claim 3, wherein the TGF-beta inhibitor is RepSox, the Ras mutant is hRasG12V, and the p53 mutant is p53DD.

5. The method of claim 3, wherein the fibroblasts are reprogrammed with one or more of the transcription factors Lhx3, Ascl1, Brn2, Myt1l, Isl1, Ngn2 and NeuroD1.

6. The method of claim 1, wherein the prospective treatments in step (ii) are selected from a library of known treatments for the neurodegenerative disorder.

7. The method of claim 1, wherein one or more of the prospective treatments in step (ii) include treatment by inhibition of PIKFYVE.

8. The method of any claim 1, wherein one or more of the prospective treatments in step (ii) include administration of a PIKFYVE inhibitor.

9. The method of claim 1, wherein two or more of the prospective treatments in step (ii) include administration of a PIKFYVE inhibitor.

10. The method of claim 1, wherein step (iii) comprises longitudinal tracking of individual motor neurons.

11. The method of claim 1, wherein step (iii) comprises (a) measuring protein localization, (b) determining the survival rate of motor neurons by imaging, or (c) any combination of any of the foregoing.

12. The method of claim 1, wherein step (iii) comprises measuring protein localization by fluorescence microscopy or fractionation.

13. The method of claim 1, wherein step (iii) comprises determining the survival rate of motor neurons following each prospective treatment.

14. The method of claim 1, further comprising the step of (iv) identifying one or more suitable treatments based on the assessment in step (iii).

15. The method of claim 14, further comprising the step of (v) treating the patient with an identified suitable treatment from step (iv).

16. (canceled)

17. (canceled)

18. The method of claim 1, wherein the fibroblasts are obtained from the skin of the patient.

19. The method of claim 1, wherein the motor neurons are generated from the fibroblasts within one month of obtaining the fibroblasts from the patient.

20. A method for evaluating a prospective treatment for a patient having a neurodegenerative disorder comprising:
(i) generating motor neurons from fibroblasts obtained from a patient, wherein the fibroblasts have been reprogrammed to produce neurons in higher yield;
(ii) testing the prospective treatment against the generated motor neurons;
(iii) assessing the motor neurons following treatment; and
(iv) determining whether to treat the patient with the prospective treatment based on the assessment in step (iii).

21. The method of claim 20, wherein the prospective treatment is the subject of a clinical trial, and step (iv) includes determining whether the patient should be a subject in the clinical trial.

22. A method for selecting patients for a clinical trial involving a prospective treatment for a neurodegenerative disorder, the method comprising,
(i) for each patient, generating motor neurons from (i) induced pluripotent stem cells from somatic cells or (ii) fibroblasts obtained from the patient;
(ii) for each patient, testing the prospective treatment against the motor neurons generated from the patient's pluripotent stem cells or fibroblasts;
(iii) for each patient, assessing the motor neurons generated from the patient's pluripotent stem cells or fibroblasts following treatment; and
(iv) selecting one or more patients for the clinical trial based on the results of the assessment in step (iii).

23. The method of claim 1, wherein the neurodegenerative disorder is selected from Huntington's disease, Alzheimer's disease, dementia such as frontotemporal dementia and Lewy body dementia, Parkinson's disease, multiple sclerosis, amyotrophic lateral sclerosis (ALS), amyloid lateral sclerosis, Friedreich's ataxia, Parkinson's disease, spinal muscle atrophy, primary lateral sclerosis, progressive muscle atrophy, progressive bulbar palsy, pseudobulbar palsy, Creutzfeldt Jakob disease, corticobasal degeneration, and progressive supranuclear palsy.

24. The method of claim 1, wherein the neurodegenerative disorder is a rapidly progressive neurodegenerative disorder.
